(19) United States
(12) Patent Application Publication
(10) Pub. No.: US 2018/0120209 A1
(43) Pub. Date: May 3, 2018

(54) ASPHALT CALIBRATION METHOD AND DEVICE

(71) Applicant: Blacklidge IP, LLC, Gulfport, MS (US)

(72) Inventor: ROY BRITTANY BLACKLIDGE, Gulfport, MS (US)

(73) Assignee: Blacklidge IP, LLC, Gulfport, MS (US)

(21) Appl. No.: 15/801,976

(22) Filed: Nov. 2, 2017

Related U.S. Application Data
(60) Provisional application No. 62/416,326, filed on Nov. 2, 2016.

(51) Int. Cl. G01N 5/02; G01N 33/42

(52) U.S. Cl. CPC G01N 5/02; G01N 33/42

(57) ABSTRACT

A system for preventing the evaporation of liquids from tack coat by sealing the testing pad inside an enclosure immediately after the tack coat is applied comprising a sealed enclosure and testing pad. The weight of the testing pad before and after tack coat application is used to calculate the calibration rate of the tack coat. Thus, by sealing the liquids inside the enclosure, the proper second weight of the testing pad is obtained resulting in an accurate calibration rate and application of tack coat on asphalt. The system further includes affixing a weighted material to the testing pad prior to placing the testing pad on asphalt and spraying the tack coat during asphalt calibration to remove the need to tape the testing pad and prevent shrinkage and movement of the testing pad during application of the tack coat.

### CROSS-REFERENCES TO RELATED APPLICATION

This application claims the benefit of U.S. Provisional Application No. 62/416,326 filed on Nov. 2, 2016, which is incorporated herein by reference in its entirety.

### FIELD OF THE INVENTION

The present invention is generally directed toward a method and device for calibrating asphalt emulsion rates. More specifically, the invention is directed to a method and device for preventing evaporation of liquids during asphalt emulsion rate calibration.

### BACKGROUND OF THE INVENTION

The use of asphalt tack coats to promote the bond between old and new pavement layers is known in the art. More specifically, these coats are applied along transverse and longitudinal vertical surfaces to prevent slippage between pavement layers, and provide structure to the pavement. The proper application of an asphalt tack coat can significantly improve the bond between the pavement layers and thus reduce the tendency for the surface course layer to slide on the underlying layer under the force of accelerating or decelerating traffic.

Commonly, tack coat materials include emulsified asphalt, neat and polymer modified PG Graded binders to create anionic and cationic emulsions, and water for dilution. If the layers are not bonded together correctly, layer independence creates reduced fatigue life, increased rutting, slippage and shoving of the asphalt. The proper application rate varies depending on the dilution and type of surface being treated, making the application rate essential to proper coverage.

In the prior art, such as ASTM D2995, the weight of the tack coat on the asphalt must be obtained almost immediately after spraying the asphalt to ensure the accurate weight is obtained. For instance, depending on ambient conditions, tack coat may fully set in 5-7 minutes meaning that virtually all water has evaporated and the weight would be less at the time of weight measurement than immediately after application. Evaporation of the liquids from tack coat reduces the weight of the tack coat and can be misinterpreted as missing tack, and thus, the application rate of the tack coat on the asphalt too low. Tack can also be lost during transportation of the testing pad when it is removed from the asphalt and transported to the scale, due to the weight of the tack on the pad causing the pad to lose its rigidity and shape. To address this issue, the California Department of Transportation teaches carefully folding the edges of the calibration pads towards the center and rolling the pad up prior to weighing it so that no emulsion is lost when the pad is removed from the asphalt. California Department of Transportation, Bonded Wearing Course, Maintenance Technical Advisory Guide (MTAG) Volume I—Flexible Pavement Preservation Second Edition, Chapter 11.

Additional issues arise when the testing pads are placed on the asphalt and secured via tape prior to application of the tack coat, as taught by the prior art. The asphalt distributor to be calibrated is driven over the testing pads while spraying tack, and then the pads are removed from the roadway and reweighed. The testing pads are often moved when the asphalt distributor passes over them and can shrink when sprayed with the tack coat.

### SUMMARY OF THE INVENTION

Water and other liquids rapidly evaporate from tack coat applied to asphalt preventing the accurate weights from being obtained and calculated when using asphalt tack coats to promote the bond between old and new pavement layers. The presently disclosed method and device prevents evaporation of liquids from the tack coat by sealing the testing pad inside an enclosure immediately after the tack coat is applied comprising a sealed enclosure and testing pad. The weight of the testing pad before and after tack coat application is used to calculate the calibration rate of the tack coat. Thus, by sealing the liquids inside the enclosure, the proper second weight of the testing pad is obtained resulting in an accurate calibration rate and application of tack coat on asphalt.

The presently disclosed system also includes a method and device which prevents the testing pad from shrinking and being disturbed by the asphalt distributor during asphalt calibration. By attaching a testing pad to a weighted piece of material similar in size to the testing pad to weigh down the calibration pad prior to application of tack, the testing pad remains stagnant on the asphalt and does not shrink.

### DETAILED DESCRIPTION

The following detailed description is presented to enable any person skilled in the art to make and use the invention. For purposes of explanation, specific details are set forth to provide a thorough understanding of the present invention. However, it will be apparent to one skilled in the art that these specific details are not required to practice the invention. Descriptions of specific applications are provided only as representative examples. Various modifications to the preferred embodiments will be readily apparent to one skilled in the art, and the general principles defined herein may be applied to other embodiments and applications without departing from the scope of the invention. The present invention is not intended to be limited to the embodiments shown, but is to be accorded the widest possible scope consistent with the principles and features disclosed herein.

Water and other liquids rapidly evaporate from tack coat laid on asphalt during the asphalt calibration process preventing the accurate weight of the tack coat from being obtained and calculated. The evaporated water and other liquids can be misinterpreted as missing tack meaning that the application rate of the tack coat on the asphalt is too low. For instance, the California Department of Transportation attempts to prevent evaporation and loss of emulsion by carefully folding the edges of the calibration testing pads towards the center and rolling the pad up prior to weighing. However, this method does not adequately prevent loss of emulsion. Therefore, a system which prevents evaporation from the tack coat is needed in the art. The system disclosed herein prevents evaporation of liquids from the surface of tack coat and can be used by inspectors to show that the tack coat is being applied at the necessary rate.

In an embodiment of the system, the device consists of an enclosure capable of sealing a testing pad immediately after the tack coat is applied. Specifically, prior to application of the tack coat, a testing pad of uniform size is placed on a scale and weighed. The initial weight of the tack coat is recorded. Next, the testing pad is laid down and secured to the surface of asphalt, such as asphalt on a road, and a thin layer of tack coat is applied to the testing pad. The tack coat is applied by a sprayer mechanism on top of the testing pad.

Immediately after spraying of the tack, the testing pad is placed inside a sealable enclosure, sealed and weighed. The weight of the testing pad is then recorded so that the application rate can be calculated. The purpose of the enclosure is to ensure that the liquids do not evaporate off the tack coat before the second weight is obtained. The first and second weights of the testing pads are used to calculate the calibration rates.

The preferred material of the testing pad is mesh but any material capable of adhering to tack coat is contemplated by this system.

A polypropylene bag is the preferred material of the sealable enclosure but any enclosure capable of sealing the testing pad and preventing evaporation, such as a container, is contemplated by this system.

To calculate the application rate, the operator must know the type of material being used and whether the emulsion has been diluted or not. In addition, he or she must know what the residual asphalt content is supposed to be and then back-calculate to determine the application rate of the emulsion from the distributor. The application rate is calculated by multiplying the residual asphalt content by a factor to determine the required application rate of a diluted emulsion. Thus, if the weight of the tack was improperly recorded after evaporation of liquids from the testing pad, the application rate will be too low.

For example, for an emulsion that is diluted 1:1 with additional water, for a required residual asphalt content of 0.05 gal./sq. yd., it would be necessary to apply the diluted asphalt emulsion at a rate of 0.15 gal./sq. yd. from the asphalt distributor. This application rate is calculated by multiplying the residual asphalt content by a factor of 3.0 to determine the required application rate of the 1:1 diluted emulsion. Similarly, if the required residual asphalt content is 0.04 gal./sq. yd., the application rate of the diluted emulsion would be 3 times as much, or 0.12 gal./sq. yd. However, if the content is recorded in this scenario as 0.03 gal/sq. yd. instead of 0.04 gal./sq. yd., the application rate would be calculated to be 0.09 gal./sq. yd. instead of 0.12 gal./sq. yd. and the tack coat would be applied at the lower rate resulting in decreased bond between the pavement layers. Inadequate bond between the pavement layers fails to reduce the tendency for the surface course layer to slide on the underlying layer under the force of accelerating or decelerating traffic.

In another embodiment of the system, prior to laying the testing pad on the asphalt, the testing pad is attached to a piece of material similar in size or the same size as the testing pad to weigh down the testing pad during spraying and prevent the testing pad from shrinking. For example, if a 1 ft by 1 ft square calibration pad is used, the attached weighted material should be approximately 1 ft by 1 ft. It is contemplated that commercially available off the shelf laminate flooring pieces can be used as the material to weigh down the testing pad. Again, for example, if a 1 ft by 1 ft square texting pad is used, a 1 ft by 1 ft square testing pad can be affixed to the testing pad prior to placement on the asphalt and spraying of the tack coat. By affixing the testing pad to a weighted material, the need to tape the testing pad to the asphalt is eliminated and the possibility that the pad will shrink or move on the asphalt is decreased.

In another embodiment of the system, asphalt shingles are affixed to the testing pad and placed asphalt side down on the asphalt to prevent slippage and shrinkage during tack coat application.

The terms “comprising,” “including,” and “having,” as used in the claims and specification herein, shall be considered as indicating an open group that may include other elements not specified. The terms “a,” “an,” and the singular forms of words shall be taken to include the plural form of the same words, such that the terms mean that one or more of something is provided. The term “one” or “single” may be used to indicate that one and only one of something is intended. Similarly, other specific integer values, such as “two,” may be used when a specific number of things is intended. The terms “preferably,” “preferred,” “prefer,” “optionally,” “may,” and similar terms are used to indicate that an item, condition or step being referred to is an optional (not required) feature of the invention.

The invention has been described with reference to various specific and preferred embodiments and techniques. However, it should be understood that many variations and modifications may be made while remaining within the spirit and scope of the invention. It will be apparent to one of ordinary skill in the art that methods, devices, device elements, materials, procedures and techniques other than those specifically described herein can be applied to the practice of the invention as broadly disclosed herein without resort to undue experimentation. All art-known functional equivalents of methods, devices, device elements, materials, procedures and techniques described herein are intended to be encompassed by this invention. Whenever a range is disclosed, all subranges and individual values are intended to be encompassed. This invention is not to be limited by the embodiments disclosed, including any shown in the drawings or exemplified in the specification, which are given by way of example and not of limitation.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

All references throughout this application, for example patent documents including issued or granted patents or equivalents, patent application publications, and non-patent literature documents or other source material, are hereby incorporated by reference herein in their entireties, as though individually incorporated by reference, to the extent each reference is at least partially not inconsistent with the disclosure in the present application (for example, a reference that is partially inconsistent is incorporated by reference except for the partially inconsistent portion of the reference).

I claim:

1. A method for calibrating asphalt emulsion rates comprising:
a. weighing a testing pad and recording the initial weight of the testing pad;
b. laying the testing pad down on the surface of the asphalt;
c. applying a thin layer of tack coat to the testing pad;
d. placing the testing pad in a sealable enclosure immediately after spraying of the tack coat and sealing the enclosure;
e. weighing the testing pad; and
f. calculating the emulsion rate.

2. The sealable enclosure of claim 1 wherein the sealable enclosure is a bag.

3. The sealable enclosure of claim 2 wherein the bag is polypropylene material.

4. The sealable enclosure of claim 1 wherein the sealable enclosure is a container.

5. A method for preventing shrinkage and movement of testing pads during asphalt calibration comprising:
a. affixing a weighted material to the testing pad prior to placing it on the asphalt; and
b. placing the testing pad weighted material side down on the asphalt prior to applying the tack coat.

6. The weighted material of claim 5 wherein the weighted material is a laminate tile of similar size as the testing pad.

7. The weighted material of claim 5 wherein the weighted material is an asphalt shingle.
